(19) United States
(12) Patent Application Publication
(10) Pub. No.: US 2012/0107381 A1
(43) Pub. Date: May 3, 2012

(54) POWDER FORMULATIONS OF POTASSIUM-BINDING ACTIVE AGENTS

(75) Inventors: Dasharatha Reddy, San Ramon, CA (US); George Tyson, Los Altos Hills, CA (US)

(73) Assignee: RELYPSA, INC., Santa Clara, CA (US)

(21) Appl. No.: 13/319,792

(22) PCT Filed: May 13, 2010
(86) PCT No.: PCT/US10/34709

§ 371 Date: Dec. 6, 2011

Related U.S. Application Data
(60) Provisional application No. 61/178,012, filed on May 13, 2009.

(51) Int. Cl. A01N 25/00; A61K 31/74; A61K 9/00

(52) U.S. Cl. 424/405; 424/400; 424/78.08

(57) ABSTRACT

The present invention is directed to powder formulations of a potassium-binding active agent, a suspending agent, and a glidant; optionally, other excipients can be added to the formulations. These formulations can be in a powder form and are useful to bind potassium in the gastrointestinal tract.

### FIELD OF THE INVENTION

The present invention is directed to powder formulation of a potassium-binding active agent, a suspending agent, a glidant, and/or an antimicrobial agent; optionally, an opacity agent can be added to the compositions. These formulations are useful to bind potassium in the gastrointestinal tract.

### BACKGROUND OF THE INVENTION

Potassium (K⁺) is one of the most abundant intracellular cations. Potassium homeostasis is maintained predominantly through the regulation of renal excretion. Various medical conditions, such as decreased renal function, genitourinary disease, cancer, severe diabetes mellitus, congestive heart failure and/or the treatment of these conditions can lead to or predispose patients to hyperkalemia. Hyperkalemia can be treated with various potassium-binding polymers including polyfluoroacrylic acid (polyFAA) as disclosed in WO 2005/097081.

Various polystyrene sulfonate potassium-binding polymers (e.g., Kayexalate®, Argamate®, Kionex®) have been used to treat hyperkalemia in patients. These polymers and polymer formulations are known to have patient compliance issues, including dosing size and frequency, taste and/or texture, and gastric irritation. For example, in some patients, constipation develops, and sorbitol is thus commonly co-administered to avoid constipation, but this leads to diarrhea and other gastrointestinal side effects. One formulation for such polystyrene sulfonate polymers is in the form of a gel in U.S. Pat. No. 6,703,013.

It has been found that powder formulations of potassium-binding active agents of the present invention are essentially tasteless when added to food and provide a comfortable mouth-feel, while minimizing the bulk of the pharmaceutical composition.

### SUMMARY OF THE INVENTION

The present invention provides a powder formulation comprising a potassium-binding active agent, a suspending agent, a glidant, or an antimicrobial agent and water; optionally, an opacity agent can be added to the compositions.

One of the aspects of the invention is a powder formulation comprising a potassium-binding polymer, water, a suspending agent and an antimicrobial agent. In this formulation, the amount of water does not prevent the powder from freely flowing.

Another aspect is a powder formulation comprising a potassium-binding active agent, a suspending agent, and a glidant. In this formulation, at least about 40 wt. % active agent is present in the composition based on the total weight of the solid components.

In some of the various aspects, the potassium-binding active agent or potassium-binding polymer comprises a crosslinked aliphatic carboxylic polymer.

### DETAILED DESCRIPTION

The present invention is directed to powder formulations comprising a potassium-binding polymer, water, a suspending agent and an antimicrobial agent, wherein the amount of water does not prevent the powder from freely flowing. The present invention also is directed toward a powder formulation comprising a potassium-binding active agent, a suspending agent, and a glidant, wherein at least about 40 wt. % active agent is present in the composition based on the total weight of the formulation. The powder formulations may additionally comprise colorants, flavors, stabilizers, or other excipients. These powder formulations are useful to bind potassium in the gastrointestinal tract to treat hyperkalemia or the risk of hyperkalemia. These formulations are advantageous in terms of their suitability for a wide range of delivery methods. For example, the compositions can be placed in food, liquid, or another appropriate delivery agent without affecting its taste or texture.

In general, the powder formulations of the present invention comprise a potassium-binding active agent; a suspending agent; and either a glidant; or an anti-microbial agent and water. In some instances, the powder formulations of the present invention comprise a potassium-binding active agent, stabilizer, suspending agent and glidant. The powder formulations of the present invention also comprise a potassium-binding active agent, stabilizer, suspending agent, glidant, water and anti-microbial agent. In other instances, the powder formulations of the present invention comprise a potassium-binding polymer, suspending agent, water and anti-microbial agent. Also, the powder formulations of the present invention comprise a potassium-binding polymer, suspending agent, glidant, water and anti-microbial agent. The powder formulations of the present invention also comprise a potassium-binding polymer, suspending agent, glidant, stabilizer, water and anti-microbial agent. The powder formulations may additionally comprise colorants, flavors, or other excipients.

In some embodiments, the potassium-binding active agent comprises a crown ether, a crown-ether like molecule, or a potassium-binding polymer. Crown ethers show selectivity for certain alkali metals over others, based on the hole-size and the size of the metal ion. See Tables 1-3 and Pedersen, C. J. 1987. Charles J. Pederson—Nobel Lecture. The discovery of crown ethers. In Nobel Lectures, Chemistry 1981-1990. T. Frangsmyr, editor. World Scientific Publishing Co., Singapore.

TABLE 1
Diameters of holes in Sample Crown Ethers, in Angstrom units

| Macrocyclic Polyethers | Diameters |
|---|---|
| All 14-crown-4 | 1.2-1.5 |
| All 15-crown-5 | 1.7-2.2 |
| All 18-crown-6 | 2.6-3.2 |
| All 21-crown-7 | 3.4-4.3 |

TABLE 2
Complexable cations and their diameters in Angstrom units

| Group I | | Group II | | Group III | | Group IV | |
|---|---|---|---|---|---|---|---|
| Li | 1.36 | | | | | | |
| Na | 1.94 | | | | | | |
| K | 2.66 | Ca | 1.98 | | | | |
| Cu(I) | 1.92 | Zn | 1.48 | | | | |
| Rb | 2.94 | Sr | 2.26 | | | | |
| Ag | 2.52 | Cd | 1.94 | | | | |
| Cs | 3.34 | Ba | 2.68 | La | 2.30 | | |
| Au(I) | 2.88 | Hg(II) | 2.20 | TI(I) | 2.80 | Pb(II) | 2.40 |
| Fr | 3.52 | Ra | 2.80 | | | | |
| NH₄ | 2.86 | | | | | | |

TABLE 3
Relative binding of sample alkali metal ions by sample crown ethers

| Polyether | Li⁺ | Na⁺ | K⁺ | Cs⁺ |
|---|---|---|---|---|
| Dicyclohexyl-14-crown-4 | 1.1 | 0 | 0 | 0 |
| Cyclohexyl-15-crown-5 | 1.6 | 19.7 | 8.7 | 4.0 |
| Dibenzo-18-crown-6 | 0 | 1.6 | 25.2 | 5.8 |
| Dicyclohexyl-18-crown-6 | 3.3 | 25.6 | 77.8 | 44.2 |
| Dicyclohexyl-21-crown-7 | 3.1 | 22.6 | 51.3 | 49.7 |
| Dicyclohexyl-24-crown-8 | 2.9 | 8.9 | 20.1 | 18.1 |

Suitable crown ethers are commercially available from Sigma-Aldrich or can be prepared by the method described in Charles J. Pedersen (1988), “Macrocyclic Polyethers: Dibenzo-18-Crown-6 Polyether and Dicyclohexyl-18-Crown-6 Polyether”. Org. Synth.; Coll. Vol. 6: 395.

In a particular embodiment, the potassium-binding active agent is a potassium binding polymer comprising units corresponding to Formulae 1 and 2, Formulae 1 and 3, and/or Formulae 1, 2, and 3:

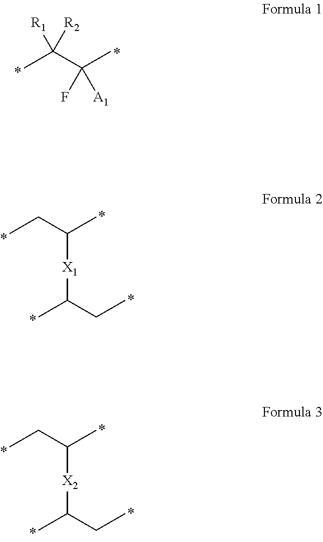

wherein R₁ and R₂ are independently selected from hydrogen, alkyl, cycloalkyl, or aryl; A₁ is a carboxylic, phosphonic, or phosphoric moiety in its salt or acid form; X₁ is an arylene linking moiety; and X₂ is an alkylene, ether, or amide linking moiety. In various embodiments, the potassium-binding polymer is a crosslinked aliphatic carboxylic acid polymer wherein R₁ and R₂ are independently selected from hydrogen, alkyl, or cycloalkyl; Aₗ is carboxylic; X₁ is an arylene linking moiety; and X₂ is an alkylene, ether, or amide linking moiety.

When X₂ is an ether linking moiety or an amide linking moiety, the ether linking moiety is —(CH₂)_{d}—O—(CH₂)ₑ— or —(CH₂)_{d}—O—(CH₂)ₑ—O—(CH₂)_{d}—, and the amide linking moiety is —C(O)—NH—(CH₂)ₚ—NH—C(O)—, d and e are independently an integer of 1 through 5, and p is an integer of 1 through 8. More specifically, d is an integer from 1 to 2, e is an integer from 1 to 3, and p is an integer of 4 to 6. The unit corresponding to Formula 2 can be derived from a difunctional crosslinking monomer having the formula CH₂═CH—X₁—CH═CH₂ wherein X₁ is as defined in connection with Formula 2. Further, the unit corresponding to Formula 3 is derived from a difunctional crosslinking monomer having the formula CH₂=CH—X₂—CH═CH₂ wherein X₂ is as defined in connection with Formula 3.

In connection with Formula 1, in one embodiment, R₁ and R₂ are hydrogen. Also, Aₗ comprises a carboxylic moiety. In connection with Formula 2, in one embodiment, X₁ is phenylene. In connection with Formula 3, in one embodiment, X₂ is ethylene, propylene, butylene, pentylene, or hexylene; more specifically, X₂ is butylene. In one specific embodiment, R₁ and R₂ are hydrogen, Aₗ is carboxylic acid, X₁ is phenylene and X₂ is butylene.

In one embodiment, the crosslinked cation exchange polymer comprises at least about 80 wt. %, particularly at least about 85 wt. %, and more particularly at least about 90 wt. % or from about 80 wt. % to about 95 wt. %, from about 85 wt. % to about 95 wt. %, from about 85 wt. % to about 93 wt. % or from about 88 wt. % to about 92 wt. % of structural units corresponding to Formula 1 based on the total weight of the structural units as used in the polymerization mixture corresponding to (i) Formulae 1 and 2, (ii) Formulae 1 and 3, or (iii) Formulae 1, 2, and 3. Additionally, the polymer can comprise a unit of Formula 1 having a mole fraction of at least about 0.87 or from about 0.87 to about 0.94 or from about 0.90 to about 0.92 based on the total number of moles of the units corresponding to (i) Formulae 1 and 2, (ii) Formulae 1 and 3, or (iii) Formulae 1, 2, and 3.

In one embodiment, the polymer contains structural units of Formulae 1, 2, and 3 and has a weight ratio of the structural unit corresponding to Formula 2 to the structural unit corresponding to Formula 3 of from about 4:1 to about 1:4, from about 2:1 to 1:2, or about 1:1. Additionally, this polymer can have a mole ratio of the structural unit of Formula 2 to the structural unit of Formula 3 of from about 0.2:1 to about 7:1, from about 0.2:1 to about 3.5:1; from about 0.5:1 to about 1.3:1, from about 0.8 to about 0.9, or about 0.85:1.

Typically, the crosslinked potassium-binding polymer comprises units corresponding to (i) Formulae 1A and 2A, (ii) Formulae 1A and 3A, or (iii) Formulae 1A, 2A, and 3A, wherein Formulae 1A, 2A and 3A are generally represented by the following structures.

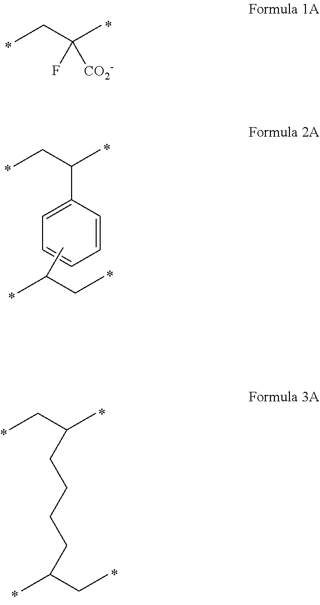

In Formula 1A, the carboxylic acid is preferably in the salt form (i.e., balanced with a counter-ion such as Ca²⁺, Mg²⁺, Na⁺, NH₄⁺, and the like). Preferably, the carboxylic acid is in the salt form and balanced with a Ca²⁺ counterion. When the carboxylic acid of the crosslinked potassium-binding polymer form is balanced with a divalent counterion, two carboxylic acid groups can be associated with the one divalent cation.

The structural units of the terpolymer can have specific ratios, for example, wherein the structural units corresponding to Formula 1A constitute at least about 85 wt. % or from about 80 to about 95 wt. %, from about 85 wt. % to about 93 wt. %, or from about 88 wt. % to about 92 wt. % based on the total weight of structural units of Formulae 1A, 2A, and 3A, calculated based on the amounts of monomers of Formulae 11A, 22A, and 33A used in the polymerization reaction, and the weight ratio of the structural unit corresponding to Formula 2A to the structural unit corresponding to Formula 3A is from about 4:1 to about 1:4, or about 1:1. Further, the ratio of structural units when expressed as the mole fraction of the structural unit of Formula 1A in the polymer is at least about 0.87 or from about 0.87 to about 0.94, or from about 0.9 to about 0.92 based on the total number of moles of the structural units of Formulae 1A, 2A, and 3A calculated from the amount of monomers of Formulae 11A, 22A, and 33A used in the polymerization reaction, and the mole ratio of the structural unit of Formula 2A to the structural unit of Formula 3A is from about 0.2:1 to about 7:1, from about 0.2:1 to about 3.5:1, from about 0.5:1 to about 1.3:1, from about 0.8:1 to about 0.9:1, or about 0.85:1.

The polymers described herein are generally random polymers wherein the exact order of the structural units of Formulae 1, 2, or 3 (derived from monomers of Formulae 11, 22, or 33), or 1A, 2A, or 3A (derived from monomers of Formulae 11A, 22A, or 33A) is not predetermined.

A potassium-binding polymer derived from monomers of Formulae 11, 22, and 33, followed by hydrolysis, can have a structure represented as follows:

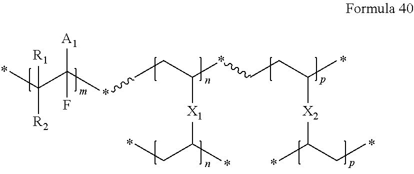

wherein R₁, R₂, A₁, X₁, and X₂ are as defined in connection with Formulae 1, 2, and 3 and m is in the range of from about 85 to about 93 mol %, n is in the range of from about 1 to about 10 mol % and p is in the range of from about 1 to about 10 mol %. The wavy bonds in the polymer structures of Formula 40 are included to represent the random attachment of structural units to one another wherein the structural unit of Formula 1 can be attached to another structural unit of Formula 1, a structural unit of Formula 2, or a structural unit of Formula 3; the structural units of Formulae 2 and 3 have the same range of attachment possibilities.

Using a polymerization process, with monomers generally represented by Formulae 1 1A, 22A and 33A, followed by hydrolysis and calcium ion exchange, a polymer represented by the general structure shown below is obtained:

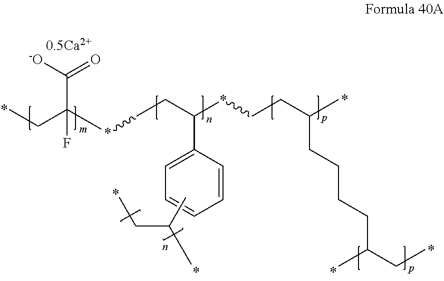

wherein m is in the range of from about 85 to about 93 mol %, n is in the range of from about 1 to about 10 mol % and p is in the range of from about 1 to about 10 mol %. The wavy bonds in the polymer structures of Formula 40A are included to represent the random attachment of structural units to one another wherein the structural unit of Formula 1A can be attached to another structural unit of Formula 1A, a structural unit of Formula 2A, or a structural unit of Formula 3A; the structural units of Formulae 2A and 3A have the same range of attachment possibilities.

Further, the crosslinked potassium-binding polymer can be the reaction product of a polymerization mixture that is subjected to polymerization conditions. The polymerization mixture may also contain components that are not chemically incorporated into the polymer. More specifically, the crosslinked potassium-binding polymer can be the reaction product of a polymerization initiator and a monomer of (i) Formulae 11 and 22, (ii) Formulae 11 and 33, or (iii) Formulae 11, 22, and 33. The monomer of Formulae 11, 22, and 33 are generally represented by

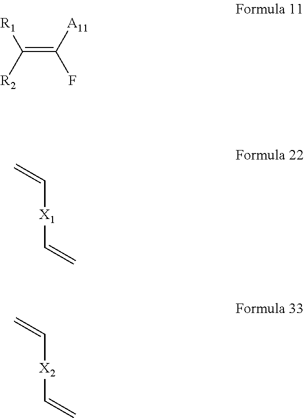

wherein R₁ and R₂ are as defined in connection with Formula 1, X₁ is as defined in connection with Formula 2, X₂ is as defined in connection with Formula 3, and A₁₁ is a protected carboxylic, phosphonic, or phosphoric containing moiety. The reaction product of a polymerization initiator and a monomer of (i) Formulae 11 and 22, (ii) Formulae 11 and 33, or (iii) Formulae 11, 22, and 33 comprises a polymer having protected acid groups and comprising units corresponding to Formula 10 and units corresponding to Formulae 2 and 3. The polymer having protected acid groups can be hydrolyzed to form a polymer having unprotected acid groups and comprising units corresponding to Formulae 1, 2, and 3. The structural units generally represented by Formula 10 have the structure

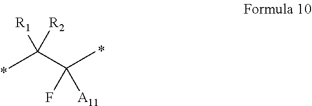

wherein R₁, R₂, and A₁₁ are as defined in connection with Formula 11.

In one embodiment, the reaction mixture comprises at least about 80 wt. %, particularly at least about 85 wt. %, and more particularly at least about 90 wt. % or from about 80 wt. % to about 95 wt. %, from about 85 wt. % to about 95 wt. %, from about 85 wt. % to about 93 wt. % or from about 88 wt. % to about 92 wt. % of monomers corresponding to Formula 11 based on the total weight of the monomers corresponding to (i) Formulae 11 and 22, (ii) Formulae 11 and 33, or (iii) Formulae 11, 22, and 33. Additionally, the reaction mixture can comprise a unit of Formula 11 having a mole fraction of at least about 0.87 or from about 0.87 to about 0.94 based on the total number of moles of the monomers corresponding to (i) Formulae 11 and 22, (ii) Formulae 11 and 33, or (iii) Formulae 11, 22, and 33.

In one embodiment, the reaction mixture contains monomers of Formulae 11, 22, and 33 and has a weight ratio of the monomer corresponding to Formula 22 to the monomer corresponding to Formula 33 from about 4:1 to about 1:4; from about 2:1 to 1:2; or about 1:1, respectively. Additionally, this mixture can have a mole ratio of the monomer of Formula 22 to the monomer of Formula 33 from about 0.2:1 to about 7:1, from 0.2:1 to 3.5:1, from about 0.5:1 to about 1.3:1, from about 0.8:1 to about 0.9:1, or about 0.85:1.

Particular crosslinked potassium-binding polymers are the reaction product of a polymerization initiator and a monomer of (i) Formulae 11 and 22, (ii) Formulae 11 and 33, or (iii) Formulae 11, 22, and 33. The monomers are generally represented by Formulae 11A, 22A, and 33A

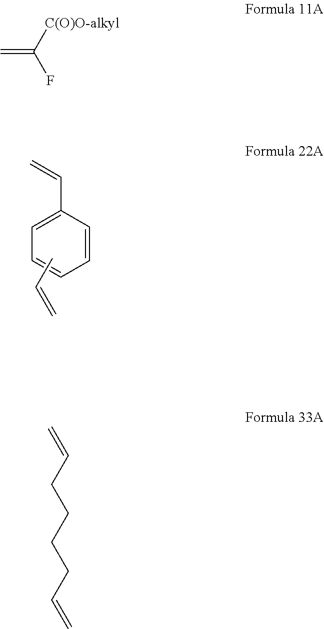

wherein alkyl is selected from methyl, ethyl, propyl, iso-propyl, butyl, iso-butyl, sec-butyl, tert-butyl, pentyl, iso-pentyl, sec-pentyl, or tert-pentyl. Preferably, the alkyl group is methyl or tert-butyl.

Further, the reaction mixture contains at least about 80 wt. %, particularly, at least about 85 wt. %, and more particularly at least about 90 wt. % or from about 80 wt. % to about 95 wt. %, from about 85 wt. % to about 95 wt. %, from about 85 wt. % to about 93 wt. % or from about 88 wt. % to about 92 wt. % of monomers corresponding to Formula 11A based on the total weight of the monomers which are generally represented by (i) Formulae 11A and 22A, (ii) Formulae 11A and 33A, or (iii) Formulae 11A, 22A, and 33A. Additionally, the reaction mixture can comprise a unit of Formula 11A having a mole fraction of at least about 0.87 or from about 0.87 to about 0.94 or from about 0.9 to about 0.92 based on the total number of moles of the monomers present in the polymer which are generally represented by (i) Formulae 11A and 22A, (ii) Formulae 11A and 33A, or (iii) Formulae 11A, 22A, and 33A.

Typically, the reaction mixture contains monomers of Formulae 11, 22, and 33 and the weight ratio of the monomer generally represented by Formula 22A to the monomer generally represented by Formula 33A is from about 4:1 to about 1:4 or about 1:1, respectively. Also, this mixture has a mole ratio of the monomer of Formula 22A to the monomer of Formula 33A of from about 0.2:1 to about 7:1, from about 0.2:1 to about 3.5:1, from about 0.5:1 to about 1.3:1, from about 0.8:1 to about 0.9:1, or about 0.85:1.

In some embodiments, an organic phase of methyl 2-fluoroacrylate (90 wt. %), 1,7-octadiene (5 wt. %) and divinylbenzene (5 wt. %) is prepared and 1 wt. % of lauroyl peroxide is added to initiate the polymerization reaction. Additionally, an aqueous phase of water, polyvinyl alcohol, phosphates, sodium chloride, and sodium nitrite is prepared. Under nitrogen and while keeping the temperature below about 30° C., the aqueous and organic phases are mixed together. Once mixed completely, the reaction mixture is gradually heated with continuous stirring. After the polymerization reaction is initiated, the temperature of the reaction mixture is allowed to rise up to about 95° C. Once the polymerization reaction is complete, the reaction mixture is cooled to room temperature and the aqueous phase is removed. The solid can be isolated by filtration once water is added to the mixture. The filtered solid is washed with water and then with a methanol/water mixture. The resulting product is a crosslinked (methyl 2-fluoroacrylate)-divinylbenzene-1,7-octadiene terpolymer.

For the hydrolysis of the polymer having ester groups to form a polymer having carboxylic acid groups, preferably, the polymer is hydrolyzed with a strong base (e.g., NaOH or KOH) to remove the alkyl (e.g., methyl) group and form the carboxylate salt. Preferably, the (methyl 2-fluoroacrylate)-divinylbenzene-1,7-octadiene terpolymer is hydrolyzed with an excess of aqueous sodium hydroxide solution at a temperature from about 30° C. to about 100° C. to yield (sodium 2-fluoroacrylate)-divinylbenzene-1,7-octadiene terpolymer. Typically, the hydrolysis reaction is carried out for about 15 to 25 hours. After hydrolysis, the solid is filtered and washed with water.

The cation of the polymer salt formed in the hydrolysis reaction depends on the base used in that step. For example, when sodium hydroxide is used as the base, the sodium salt of the polymer is formed. This sodium ion can be exchanged for another cation by contacting the sodium salt with an excess of an aqueous metal salt to yield an insoluble solid of the desired polymer salt. After the desired ion exchange, the product is washed with an alcohol and water and dried directly or dried after a dewatering treatment with denatured alcohol. For example, the sodium salt of the potassium-binding polymer is converted to the calcium salt by washing with a solution that substitutes calcium for sodium, for example, by using calcium chloride, calcium acetate, calcium lactate gluconate, or a combination thereof. And, more specifically, to exchange sodium ions for calcium ions, the (sodium 2-fluoroacrylate)-divinylbenzene-1,7-octadiene terpolymer is contacted with an excess of aqueous calcium chloride to yield an insoluble solid of crosslinked (calcium 2-fluoroacrylate)-divinylbenzene-1,7-octadiene terpolymer.

In some embodiments, the potassium-binding polymer useful for treating hyperkalemia may be a resin having the physical properties discussed herein (including particle shape and size) and comprising polystyrene sulfonate cross linked with divinyl benzene. Various resins having this structure are available from The Dow Chemical Company under the trade name Dowex, such as Dowex 50WX2, 50WX4 or 50WX8.

In various embodiments, a potassium-binding polymer comprises the representative structural units shown in Table 1 wherein the asterisk at the end of a bond indicates that bond is attached to another structural unit or to a crosslinking unit.

TABLE 1
Examples of potassium-binding structural units—structures and theoretical binding
capacities

| | | | Fraction of | Fraction of | Expected | |
|---|---|---|---|---|---|---|
| | Molar mass | Theoretical | titratable H @ | titratable H @ | Capacity @ | Expected Capacity @ |
| | per charge | capacity | pH 3 | pH 6 | pH3 | pH6 |
| 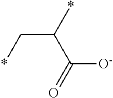 | 71 | 14.1 | 0.05 | .35 | 0.70 | 4.93 |
| 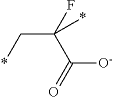 | 87 | 11.49 | 0.2 | 0.95 | 2.3 | 10.92 |
| 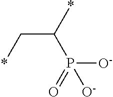 | 53 | 18.9 | 0.25 | 0.5 | 4.72 | 9.43 |
| 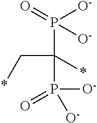 | 47.5 | 21.1 | 0.25 | 0.5 | 5.26 | 10.53 |
| 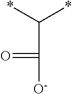 | 57 | 17.5 | 0.1 | 0.5 | 1.75 | 8.77 |
| 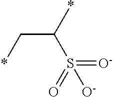 | 107 | 9.3 | 1 | 1 | 9.35 | 9.35 |
| 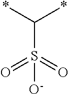 | 93 | 10.8 | 1 | 1 | 10.75 | 10.75 |
| 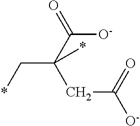 | 63 | 15.9 | 0 | 0.4 | 0 | 6.35 |
| 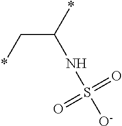 | 125 | 8 | 1 | 1 | 8 | 8 |
| 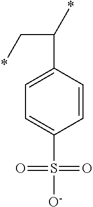 | 183 | 5.5 | 1 | 1 | 5.46 | 5.46 |
| 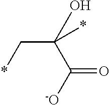 | 87 | 11.49 | .1 | .6 | 1.14 | 6.89 |

Other suitable potassium-binding polymers contain repeat units having the following structures:

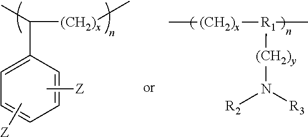

wherein R₁ is a bond or nitrogen, R₂ is hydrogen or Z, R₃ is Z or —CH(Z)₂, each Z is independently SO₃H or PO₃H, n is equal to or greater than one, x is 2 or 3, and y is 0 or 1, n is about 50 or more, more particularly n is about 100 or more, even more particularly n is about 200 or more, and most particularly n is about 500 or more.

Sulfamic (i.e. when Z═SO₃H) or phosphoramidic (i.e. when Z═PO₃H) polymers can be obtained from amine polymers or monomer precursors treated with a sulfonating agent such as sulfur trioxide/amine adducts or a phosphonating agent such as P₂O₅, respectively. Typically, the acidic protons of phosphonic groups are exchangeable with cations, like sodium or potassium, at pH of about 6 to about 7.

Suitable phosphonate monomers include vinyl phosphonate, vinyl-1,1-bis phosphonate, and ethylenic derivatives of phosphonocarboxylate esters, oligo(methylenephosphonates), and hydroxyethane-1,1-diphosphonic acid. Methods of synthesis of these monomers are well known in the art.

The potassium-binding structural units and repeat units containing acid groups as described above are crosslinked to form the crosslinked potassium-binding polymers. Representative crosslinking monomers include those shown in Table 2.

TABLE 2
Crosslinker Abbreviations and Structures

| | | | Molecular |
|---|---|---|---|
| Abbreviation | Chemical name | Structure | Weight |
| X-V-1 | ethylenebisacrylamide | 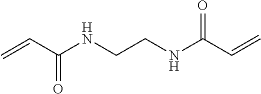 | 168.2 |
| X-V-2 | N,N′-(ethane-1,2- diyl)bis(3-(N- vinylformamido) propanamide) | 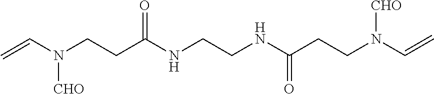 | 310.36 |
| X-V-3 | N,N′-(propane-1,3- diyl)diethenesulfonamide | 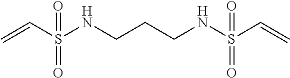 | 254.33 |
| X-V-4 | N,N′- bis(vinylsulfonylacetyl) ethylene diamine | 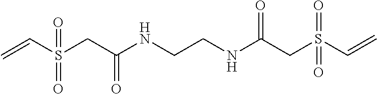 | 324.38 |
| X-V-5 | 1,3-bis(vinylsulfonyl) 2- propanol | 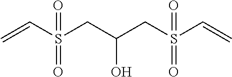 | 240.3 |
| X-V-6 | vinylsulfone | 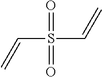 | 118.15 |
| X-V-7 | N,N′- methylenebisacrylamide | 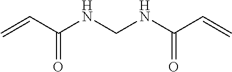 | 154.17 |
| ECH | epichlorohydrin | 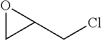 | 92.52 |
| DVB | Divinyl benzene | 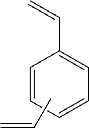 | 130.2 |
| ODE | 1,7-octadiene | 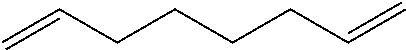 | 110.2 |
| HDE | 1,5-hexadiene | 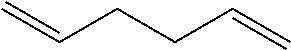 | 82.15 |

In some other embodiments, the crosslinked potassium-binding polymer includes a pKa-decreasing group, preferably an electron-withdrawing substituent, located adjacent to the acid group, preferably in the alpha or beta position of the acid group. The preferred position for the electron-withdrawing group is attached to the carbon atom alpha to the acid group. Generally, electron-withdrawing substituents are a hydroxyl group, an ether group, an ester group, an acid group, or a halide atom. More preferably, the electron-withdrawing substituent is a halide atom. Most preferably, the electron-withdrawing group is fluoride and is attached to the carbon atom alpha to the acid group. Acid groups are carboxylic, phosphonic, phosphoric, or combinations thereof

Other particular polymers result from the polymerization of alpha-fluoro acrylic acid, difluoromaleic acid, or an anhydride thereof. Monomers for use herein include α-fluoroacrylate and difluoromaleic acid, with α-fluoroacrylate being most preferred. This monomer can be prepared from a variety of routes, see for example, Gassen et al, J. Fluorine Chemistry, 55, (1991) 149-162, K F Pittman, C. U., M. Ueda, et al. (1980). Macromolecules 13(5): 1031-1036. Difluoromaleic acid is prepared by oxidation of fluoroaromatic compounds (Bogachev et al, Zhurnal Organisheskoi Khimii, 1986, 22(12), 2578-83), or fluorinated furan derivatives (See U.S. Pat. No. 5,112,993). A mode of synthesis of α-fluoroacrylate is given in EP 415214.

The active ingredient is the main component of the formulation by weight percent. In some of the various embodiments, at least about 40 wt. % of the active ingredient is present in the powder formulation based on the total weight of the formulation. In other embodiments, at least about 45 or 50 wt. % of the active ingredient is present in the powder formulation based on the total weight of the formulation. In some of these embodiments, from about 45 wt. % to about 90 wt. % of the active ingredient is present or from about 50 wt. % to about 90 wt. % of the active ingredient is present in the powder formulation based on the total weight of the formulation.

Stabilizers such as stabilizing polyols may be added in an amount sufficient to reduce the release of fluoride ion from the potassium-binding polymer upon storage as compared to an otherwise identical formulation containing no stabilizing polyol at the same temperature and storage time. The stabilizing polyol has also been observed to increase efficacy of the formulation based on fecal excretion of potassium. The stabilizing polyol is a linear polyol, selected from the group consisting of D-(+)arabitol, erythritol, glycerol, maltitol, D-mannitol, ribitol, D-sorbitol, xylitol, fructose, manitol, and mannose. In preferred embodiments, the stabilizing polyol is a sugar. In even more preferred embodiments, the stabilizing polyol is a sugar selected from the group consisting of xylitol, sorbitol, and a combination thereof. The powder formulations contain from about 10 wt. % to about 40 wt. % stabilizing polyol based on the total weight of the formulation. Preferably, the powder formulation comprises from about 15 wt. % to about 35 wt. %; from about 20 wt. % to about 40 wt. %; preferably from about 20 wt. % to about 35 wt. % stabilizing polyol based on the total weight of the formulation.

Water can also be added as a stabilizer. The moisture content of the composition can be balanced with the stabilizing polyol to provide a stabilized polymer composition. In general, as the moisture content rises, the concentration of polyol can be reduced. However, the moisture content should not rise so high as to prevent the formulation from being free flowing during manufacturing, packaging or use. It is generally assumed in the art that as the moisture content rises, the flow properties of a particle deteriorate due to agglomeration. However, for the formulations of the present invention, the powder generally flows freely despite the moisture content. In general, the moisture content can range from about 0 wt. % to about 30 wt. % based on the total weight of the formulation. More preferably, the moisture content can range from about 1.0 wt. % to about 30 wt. %, and more preferably from about 10 wt. % to about 25 wt. %; even more preferably from about 15 wt. % to about 25 wt. % based on the total weight of the formulation.

In addition, the powder formulations of the present invention include a suspending agent. The suspending agent is a substance that improves the suspension of the active ingredient in another substance, particularly an aqueous solution or soft food. Suitable suspending agents include, but are not limited to xanthan gum, polycarbophil, hydroxypropyl methyl cellulose (HPMC), povidone, methylcellulose, dextrin, sodium alginate, (poly)vinyl alcohol, microcrystalline cellulose, a colloidal silica, bentonite clay, or a combination thereof. The suspending agent can be present in a concentration ranging from about 0.25 wt. % to about 7.0 wt. %, and more specifically from about 0.3 wt. % to about 3.0 wt. % based on the total weight of formulation. A preferred suspending agent is xanthan gum. The xanthan gum can be present in a preferred powder formulation at a concentration of 0.7 wt. % based on the total weight of formulation.

In some instances, the powder formulation is free of an antimicrobial agent. In other cases, the powder formulation includes an antimicrobial agent (or preservative). The antimicrobial agent decreases the microbe growth in the powder formulation to improve its shelf-stability. Suitable antimicrobial agents include, but are not limited to α-tocopherol, ascorbate, alkylparabens (e.g., methylparaben, ethylparaben, propylbaraben, butylparaben, pentylparaben, hexylparaben, benzylparaben), chlorobutanol, phenol, sodium benzoate, benzalkonium chloride, benzethonium chloride, chlorobutanol, phenylethyl alcohol, or a combination thereof. The antimicrobial agent can be present in a concentration ranging from about 0 wt. % to about 1.5 wt. %, from about 0.05 wt. % to about 1.5 wt. % and more specifically from about 0.5 wt. % to about 1.5 wt. % based on the total weight of formulation. A preferred combination of antimicrobial agents is methylparaben and propylparaben. The concentration of the methylparaben is preferably about 0.05 wt. % to about 1.0 wt. % and the concentration of the propylparaben is preferably about 0.01 wt. % to about 0.2 wt. % based on the total weight of formulation.

Another component of the powder formulation is a glidant (or flow enhancing agent). The glidant is added to the powder to aid the flowability of the powder. Suitable glidants include, but are not limited to colloidal silicon dioxide, (e.g., Cab-O-Sil™, M5), aluminum silicate, talc, powdered cellulose, magnesium trisilicate, silicon dioxide, kaolin, glycerol monostearate, metal stearates such as magnesium stearate, titanium dioxide, starch, or a combination thereof The glidant can be present in a concentration ranging from about 0 wt. % to about 4.0 wt. %, and more specifically from about 0.1 wt. % to about 4 wt. % and even more specifically from about 0.5 wt. % to about 2 wt. % based on the total weight of formulation. A preferred glidant is colloidal silicon dioxide. The colloidal silicon dioxide is present in a preferred powder formulation at a concentration of 0.94 wt. % based on the total weight of formulation.

Optionally, an opacity agent can be added to the formulation. The opacity agent generally has a high refractive index and increases the opacity of the formulation, particularly upon mixture into a food or liquid. Suitable opacity agents include, but are not limited to titanium dioxide, zinc oxide, aluminum oxide, or a combination thereof The opacity agent can be present in a concentration ranging from about 0 wt. % to about 0.5 wt. %, and more specifically from about 0 wt. % to about 0.4 wt. % based on the total weight of formulation. A preferred opacity agent is titanium dioxide. In a preferred powder formulation, the concentration of titanium dioxide is 0.34 wt. % based on the total weight of the formulation.

An optional component of the formulations is a coloring agent. A coloring agent can be added to provide a consistent appearance to the powder. Suitable coloring agents include, but are not limited to alumina, aluminum powder, annatto extract, natural and synthetic beta-carotene, bismuth oxychloride, bronze powder, calcium carbonate, canthaxanthin, caramel, carmine, chlorophyllin, copper complex, chromium hydroxide green, chromium oxides greens, cochineal extract, copper powder, potassium sodium copper chlorophyllin (chlorophyllin copper complex), dihydroxyacetone, ferric ammonium ferrocyanide (iron blue), ferric ferrocyanide (iron blue), guanine (pearl essence), mica, mica-based pearlescent pigment, pyrophyllite, synthetic iron oxide, talc, titanium dioxide, zinc oxide, FD&C Blue #1, FD&C Blue #2, FD&C Green #3, D&C Green #5, D&C Orange #5, FD&C Red #3, D&C Red #6, D&C Red #7, D&C Red #21, D&C Red #22, D&C Red #27, D&C Red #28, D&C Red #30, D&C Red #33, D&C Red #36, FD&C Red #40, FD&C Yellow #5, FD&C Yellow #6, D&C Yellow #10, or a combination thereof. The coloring agent can be present in a concentration ranging from about 0 wt. % to about 0.1 wt. %, and more specifically from about 0 wt. % to about 0.05 wt. % based on the total weight of formulation. One example of a coloring agent is a blend of coloring agents to provide a yellow, orange, or red color, with the concentration of the blend being about 0.02 wt. % based on the total weight of the formulation. Another optional component of the compositions is a flavoring agent and/or sweetener. The flavoring agent can be added to the composition to provide an acceptable taste. Suitable flavoring agents include, but are not limited to lime, lemon, orange, vanilla, citric acid, and combinations thereof

The stabilizing polyol and water are typically added to the potassium-binding active agent before mixing with other formulation ingredients. When the active agent is a potassium-binding polymer, the salt of the polymer is slurried with an aqueous solution of polyol (e.g., sorbitol), with the slurry containing an excess amount of polyol based on polymer weight. The slurry is maintained under conditions known to those of skill in the art, such as for at least 3 hours and ambient temperatures and pressures. The solids are then filtered off and dried to desired moisture content.

The powder formulations can be prepared by thoroughly mixing a portion of the potassium-binding active agent (loaded with stabilizers) with the other excipients (e.g., suspending agent, glidant, antimicrobial agent, opacity agent, coloring agent, flavoring agent, etc.). This solid mixture is passed through a mesh screen and then the remaining portion of the active agent is added and the entire batch is mixed thoroughly, and screened again, to form the powder formulation. Alternatively, the ingredients may be mixed at once wherein the entire amount of active agent is mixed with the other excipients. There is no criticality to the order of mixing the active agent with the other excipients, or in the portions mixed. The powder formulations of the invention can be prepared by the method described above or any of the methods known in the art of pharmacy. For example, standard pharmaceutical formulation techniques such as those described in Remington's Pharmaceutical Sciences, 18th ed. (1990), Mack Publishing Company, Easton, Pa., the disclosure of which is incorporated herein by reference, can be used.

Free flowing as used herein depends on particle size, moisture and van der Waals forces, as is known in the art. In some embodiments, free flowing can be defined as the ratio of tap density divided by bulk density being less than about 1.6, and more preferably less than about 1.2. For the formulations of this invention, the ratio of tap density divided by bulk density is preferably less than about 1.1.

The particles in the formulations of this invention are generally in the form of substantially spherical particles. As used herein, the term “substantially” means generally rounded particles have an average aspect ratio of about 1.0 to about 2.0. Aspect ratio is the ratio of the largest linear dimension of a particle to the smallest linear dimension of the particle. Aspect ratios may easily be determined by those of ordinary skill in the art. This definition includes spherical particles, which by definition have an aspect ratio of 1.0. In some embodiments, the particles have an average aspect ratio of about 1.0, 1.2, 1.4, 1.6, 1.8 or 2.0. The particles may be round or elliptical when observed at a magnification wherein the field of view is at least twice the diameter of the particle.

The particles in the formulations of this invention have a mean diameter of from about 20 μm to about 200 μm. Specific ranges are where the particles have a mean diameter of from about 20 μm to about 200 μm, from about 20 μm to about 150 μm, or from about 20 μm to about 125 μm. Other ranges include from about 35 μm to about 150 μm, from about 35 μm to about 125 μm, or from about 50 μm to about 125 μm. Particle sizes, including mean diameters, distributions, etc. can be determined using techniques known to those of skill in the art. For example, U.S. Pharmacopeia (USP) <429> discloses methods for determining particle sizes.

Various particles in the formulation also have less than about 4 volume percent of the particles that have a diameter of less than about 10 μm; particularly, less than about 2 volume percent of the particles that have a diameter of less than about 10 μm; more particularly, less than about 1 volume percent of the particles that have a diameter of less than about 10 μm; and even more particularly, less than about 0.5 volume percent of the particles that have a diameter of less than about 10 μm. In other cases, specific ranges are less than about 4 volume percent of the particles that have a diameter of less than about 20 μm; less than about 2 volume percent of the particles that have a diameter of less than about 20 μm; less than about 1 volume percent of the particles that have a diameter of less than about 20 μm; less than about 0.5 volume percent of the particles that have a diameter of less than about 20 μm; less than about 2 volume percent of the particles that have a diameter of less than about 30 μm; less than about 1 volume percent of the particles that have a diameter of less than about 30 μm; less than about 1 volume percent of the particles that have a diameter of less than about 30 μm; less than about 1 volume percent of the particles that have a diameter of less than about 40 μm; or less than about 0.5 volume percent of the particles that have a diameter of less than about 40 μm. In various embodiments, the particle size distribution wherein not more than about 5 volume % of the particles have a diameter less than about 30 μm (i.e., D(0.05)<30 μm), not more than about 5 volume % of the particles have a diameter greater than about 250 μm (i.e., D(0.05)>250 μm), and at least about 50 volume % of the particles have a diameter in the range from about 70 to about 150 μm (i.e., 70 μm≧D(0.50)≧150 μm).

The particle distribution of the powder can be described as the span. The span of the particle distribution is defined as (D(0.95)−D(0.05))/D(0.5), where D(0.95) is the value wherein 95% of the particles have a diameter below that value, D(0.05) is the value wherein 5% of the particles have a diameter below that value, and D(0.5) is the value wherein 50% of the particles have a diameter above that value and 50% of the particles have a diameter below that value as measured by laser diffraction. The span of the particle distribution is typically 0.3 to 3.0, more specifically 0.5 to 1.5 and more specifically about 0.7 to 0.9 or most preferably from 1.0 to 1.3. Particle size distributions can be measured using Niro Method No. A 8 d (revised September 2005), available from GEA Niro, Denmark, using the Malvern Mastersizer.

The particles in the powder formulation may be substantially spherical with a substantially smooth surface. A substantially smooth surface is a surface wherein the average distance from the peak to the valley of a surface feature determined at random over several different surface features and over several different particles is less than about 2 μm, less than about 1 μm, or less than about 0.5 μm. Typically, the average distance between the peak and the valley of a surface feature is less than about 1 μm. The surface morphology can be measured using several techniques including those for measuring roughness. Roughness is a measure of the texture of a surface. It is quantified by the vertical deviations of a real surface from its ideal form. If these deviations are large, the surface is rough; if they are small the surface is smooth. Roughness is typically considered to be the high frequency, short wavelength component of a measured surface. For example, roughness may be measured using contact or non-contact methods. Contact methods involve dragging a measurement stylus across the surface; these instruments include profilometers and atomic force microscopes (AFM). Non-contact methods include interferometry, confocal microscopy, electrical capacitance and electron microscopy. These methods are described in more detail in Chapter 4: Surface Roughness and Microtopography by L. Mattson in Surface Characterization, ed. by D. Brune, R. Hellborg, H. J. Whitlow, O. Hunderi, Wiley-VCH, 1997.

The powder formulations may be suspended in an ingestible liquid such as water or juice. The resulting mixture has an acceptable taste, texture, and mouth-feel and therefore can be conveniently administered to a patient as a drink. The drink can be a suspension or solution. Alternatively, the disclosed powder formulation can be mixed with soft foods, such as mashed potatoes, oatmeal, apple sauce, yogurt, or pudding. In particular, the powder formulations of this invention are substantially unreactive with food, which adds to compliance enhancement (particularly for patients who are on a water restricted diet). Substantially unreactive in this context means that the powder formulations do not substantially affect the taste or consistency of the food in which it is mixed or placed. Also, “do not substantially affect the taste of the food” means that powder formulations generally will not dry the mouth or taste unacceptably gritty (e.g., like sand in the food). The powder formulations are tasteless and therefore do not adversely affect the taste of the food or liquid to which it is added.

These powder formulations can be packaged in a container, that is a non-ingestible device which can hold and preserve the stability of the powder formulation from the time of manufacture of the powder formulation to the time of consumption by patients. As described above, the powder formulation is generally free-flowing. Containers suitable for the present invention include a sachet, (e.g., a paper bag, plastic film powder bag, metal foil, etc.) a bottle (e.g., glass, plastic, or metal bottle), a tub, and an ampule. Preferably, the container of the invention is a plastic bottle. The bottles are generally non-reactive with the formulations and have a physical integrity sufficient for handling in an automated process. There is typically a good moisture barrier and a child-resistant, but elder-friendly seal. The container can also be a sachet that holds a single dose (while a bottle may hold one or more doses). The sachet preferably is easily transported and made from a durable film (e.g., polymer, metal, etc.) that resists folding, puncture and moisture. The sachet also preferably has an easily torn seal.

The container containing the powder formulation can be a unit-dose or a multi-dose container. For example, the container of the invention can contain a single dose of the powder formulation mixed with the pharmaceutically acceptable excipients, such as a single-dose bottle. Alternatively, the container of the invention can contain at least two doses of the powder formulation, such as a bottle or tub with the powder formulation from which a unit dose is measured by, e.g., a spoon or cup, or an instrument capable of dispensing a pre-defined dosage amount.

Typically the dose of the formulations described herein used to obtain the desired therapeutic and/or prophylactic benefits is about 0.5 gram/day to about 60 gram/day as measured by the amount of active ingredient in the formulation. A particular dose range is about 5 gram/day to about 60 gram/day, more particular is about 5 gram/day to about 30 gram/day, even more particularly is about 7.5 gram/day to about 30 gram/day or about 7.5 gram/day to about 15 gram/day or about 15 gram/day to about 30 gram/day as measured by the amount of active ingredient in the formulation. In various administration protocols, the dose is administered about three times a day, for example, with meals. In other protocols, the dose is administered once a day or twice a day. These doses can be for chronic or acute administration.

The methods and powder formulations described herein are suitable for removal of potassium from a patient wherein a patient is in need of such potassium removal. For example, patients suffering from hyperkalemia or at risk of hyperkalemia caused by disease and/or use of certain drugs benefit from such potassium removal. Further, patients at risk for developing high serum potassium concentrations through use of agents that cause potassium retention could be in need of potassium removal. The methods described herein are applicable to these patients regardless of the underlying condition that is precipitating the high serum potassium levels.

If necessary, the powder formulations may be administered in combination with other therapeutic agents. The choice of therapeutic agents that can be co-administered with the formulation of the invention will depend, in part, on the condition being treated.

Further, patients suffering from chronic kidney disease and heart failure can be particularly in need of potassium removal because agents used to treat these conditions may cause potassium retention in a significant population of these patients. For these patients, decreased renal potassium excretion results from renal failure (e.g., decreased glomerular filtration rate), often coupled with the ingestion of drugs that interfere with potassium excretion, e.g., potassium-sparing diuretics, angiotensin-converting enzyme inhibitors (ACEs), angiotensin receptor blockers (ARBs), beta blockers, renin inhibitors, aldosterone synthase inhibitors, non-steroidal anti-inflammatory drugs, heparin, or trimethoprim. For example, patients suffering from chronic kidney disease can be prescribed various agents that will slow the progression of the disease; for this purpose, angiotensin-converting enzyme inhibitors (ACEs), angiotensin receptor blockers (ARBs), and aldosterone antagonists are commonly prescribed as in compliance with accepted guidelines. In these treatment regimens the angiotensin-converting enzyme inhibitor is captopril, zofenopril, enalapril, ramipril, quinapril, perindopril, lisinopril, benazipril, fosinopril, or combinations thereof; the angiotensin receptor blocker is candesartan, eprosartan, irbesartan, losartan, olmesartan, telmisartan, valsartan, or combinations thereof; and the renin inhibitor is aliskiren.

For treatment of heart failure, various aldosterone antagonists can be administered to treat underlying edema or hypertension. The aldosterone antagonists can also cause potassium retention. Thus, it can be advantageous for patients in need of these treatments to also be treated with an agent that removes potassium from the body, such as the formulations herein. The aldosterone antagonists typically prescribed are spironolactone, eplerenone, and the like.

In certain particular embodiments, the powder formulations described herein can be administered on a periodic basis to treat a chronic condition. Typically, such treatments will enable patients to continue using drugs in effective amounts that may cause hyperkalemia, such as potassium-sparing diuretics, ACEs, ARBs, aldosterone antagonists, β-blockers, renin inhibitors, non-steroidal anti-inflammatory drugs, heparin, trimethoprim, or combinations thereof. Also, use of the powder formulations described herein will enable certain patient populations, who were unable to use certain above-described drugs, to use such drugs.

In certain other embodiments, the compositions and methods described herein are used in the treatment of hyperkalemia in patients in need thereof, for example, caused by excessive intake of potassium. Excessive potassium intake alone is an uncommon cause of hyperkalemia. Often, hyperkalemia is caused by indiscriminate potassium consumption in a patient with impaired mechanisms for the intracellular shift of potassium or renal potassium excretion.

In the present invention, the powder formulations can be co-administered or co-prescribed with other active pharmaceutical agents, which can include simultaneous administration of the two agents in the same dosage form, simultaneous administration in separate dosage forms, and separate administration. For example, for the treatment of hyperkalemia, the powder formulations can be co-administered with drugs that cause the hyperkalemia, such as potassium-sparing diuretics, angiotensin-converting enzyme inhibitors (ACEs), angiotensin receptor blockers (ARBs), beta blockers, renin inhibitors, non-steroidal anti-inflammatory drugs, heparin, or trimethoprim. In particular, the powder formulations can be co-administered with ACEs (e.g., captopril, zofenopril, enalapril, ramipril, quinapril, perindopril, lisinopril, benazipril, and fosinopril), ARBs (e.g., candesartan, eprosartan, irbesartan, losartan, olmesartan, telmisartan, and valsartan) and renin inhibitors (e.g. aliskiren). In particular embodiments, the agents are simultaneously administered, wherein both the agents are present in separate formulations. In other embodiments, the agents are administered separately.

The term “treating” or “treatment” as used herein includes achieving a therapeutic benefit. By therapeutic benefit is meant eradication, amelioration, or prevention of the underlying disorder being treated. For example, in a hyperkalemia patient, therapeutic benefit includes eradication or amelioration of the underlying hyperkalemia or hyperkalemia risk. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of a powder formulation comprising a potassium-binding polymer to a patient suffering from hyperkalemia provides therapeutic benefit not only when the patient's serum potassium level is decreased, but also when an improvement is observed in the patient with respect to other disorders that accompany hyperkalemia like renal failure or where a patient may remain on other drugs and/or at effective doses that increase the risk of hyperkalemia. In some treatment regimens, the powder formulations may be administered to a patient at risk of developing hyperkalemia or to a patient reporting one or more of the physiological symptoms of hyperkalemia, even though a diagnosis of hyperkalemia may not have been made.

The powder formulations of the present invention include compositions wherein the active potassium-binding active agents are present in an effective amount, i.e., in an amount effective to achieve therapeutic or prophylactic benefit. The actual amount effective for a particular application will depend on the patient (e.g., age, weight, etc.), the condition being treated, and the route of administration. Determination of an effective amount is well within the capabilities of those skilled in the art, especially in light of the disclosure herein. The effective amount for use in humans can be determined from animal models. For example, a dose for humans can be formulated to achieve gastrointestinal concentrations that have been found to be effective in animals.

Unless otherwise indicated, the alkyl groups described herein are preferably lower alkyl containing from one to eight carbon atoms in the principal chain and up to 20 carbon atoms. Alkyls may be substituted or unsubstituted and straight or branched chain. Examples of unsubstituted alkyls include methyl, ethyl, n-propyl, i-propyl, n-butyl, i-butyl, s-butyl, t-butyl, n-pentyl, i-pentyl, s-pentyl, t-pentyl, and the like. The term “substituted” as in “substituted aryl,” “substituted alkyl,” and the like, means that in the group in question (i.e., the alkyl, aryl or other moiety that follows the term), at least one hydrogen atom bound to a carbon atom is replaced with one or more substituent groups such as hydroxy, alkoxy, alkylthio, phosphino, amino, halo, silyl, nitro, esters, ketones, heterocyclics, aryl, and the like. When the term “substituted” introduces a list of possible substituted groups, it is intended that the term apply to every member of that group. That is, the phrase “substituted alkyl, and aryl” is to be interpreted as “substituted alkyl and substituted aryl.” Similarly, “optionally substituted alkyl and aryl” is to be interpreted as “optionally substituted alkyl and optionally substituted aryl.”

The term “cycloalkyl” as used herein denotes optionally substituted cyclic alkyl groups containing from three to eight carbon atoms in one ring and up to 20 carbon atoms in a multiple ring group. They may be substituted or unsubstituted and include cyclopropyl, cyclobutyl, cyclopentyl, cyclohexyl, cycloheptyl, cyclooctyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “carboxylic acid” refers to a RC(O)OH compound where R can be hydrogen, or substituted or unsubstituted alkyl, alkenyl, alkynyl, aryl, substituted aryl.

The “carboxylic acid protecting groups” described herein are moieties that block reaction at the protected carboxylic acid group while being easily removed under conditions that are sufficiently mild so as not to disturb other substituents of the various compounds. For example, the carboxylic acid protecting groups associate with the oxygen of the acid group to make —C(O)OP_{g} groups wherein P_{g} can be alkyl (e.g., methyl, ethyl, n-propyl, i-propyl, n-butyl, i-butyl, s-butyl, t-butyl, n-pentyl, i-pentyl, s-pentyl, t-pentyl, and the like), benzyl, silyl (e.g., trimethylsilyl (TMS), triethylsilyl (TES), triisopropylsilyl (TIPS), triphenylsilyl (TPS), t-butyldimethylsilyl (TBDMS), t-butyldiphenylsilyl (TBDPS) and the like). A variety of protecting groups for the hydroxyl group of the carboxylic acid group and the synthesis thereof may be found in “Protective Groups in Organic Synthesis” by T. W. Greene and P. G. M. Wuts, John Wiley & Sons, 1999.

Having described the invention in detail, it will be apparent that modifications and variations are possible without departing from the scope of the invention defined in the appended claims.

### EXAMPLES

The following non-limiting examples are provided to further illustrate the present invention.

Materials for Example 1. Methyl 2-fluoroacrylate (MeFA) was purchased from SynQuest Labs. It contained 0.2 wt% hydroquinone and was vacuum distilled before use. Divinylbenzene (DVB) was purchased from Aldrich, technical grade, 80%, mixture of isomers, and was used as received. 1,7-octadiene (ODE) was purchased from Aldrich, 98%, and used as received. Lauroyl peroxide (LPO) was purchased from ACROS Organics, 99%, and used as received. Polyvinyl alcohol (PVA), typical molecular weight 85,000-146,000, 87-89% hydrolyzed, was purchased from Aldrich, and used as received. Sodium chloride (NaCl) was purchased from Aldrich and used as received. Sodium phosphate dibasic heptahydrate (Na₂HPO₄.7H₂O) was purchased from Aldrich and used as received. Sodium phosphate monobasic monohydrate (NaH₂PO₄.H₂O) was purchased from Aldrich and used as received.

### Example 1

### Synthesis of FAA Beads with DVB/ODE Weight Ratio of 5/5

The polymerization was carried out in a 1 L three-neck Morton-type round bottom flask equipped with an overhead mechanical stirrer with a Teflon paddle and a water condenser. An organic phase was prepared by mixing MeFA (54 g), DVB (3 g), ODE (3 g) and LPO (0.6 g), and an aqueous phase was prepared by dissolving PVA (3g) and NaCl (11.25 g) in water (285.75 g). The organic and aqueous phases were then mixed in the flask and stirred at 300 rpm under nitrogen. The flask was immersed in a 70° C. oil bath for 5 hours and cooled to room temperature. The internal temperature during reaction was about 65° C. The solid product was washed with water and collected by filtration. The white solid was freeze-dried, affording dry solid beads (56.91 g, 95%).

Hydrolysis was carried out in the same setup as for the polymerization. PolyMeFA beads (39.17 g) from the polymerization reaction were suspended in a NaOH solution (400 g, 10 wt. %) and stirred at 200 rpm. The mixture was heated in a 95° C. oil bath for 20 hours and cooled to room temperature. The solid product was washed with water and collected by filtration. After freeze-drying, polyFAA beads (42.31 g, 100%) were obtained.

The calcium form of the polyFAA beads was prepared by exposing the (sodium 2-fluoroacrylate)-divinylbenzene-1,7-octadiene copolymer to an excess of aqueous calcium chloride solution to yield insoluble cross-linked (calcium 2-fluoroacrylate)-divinylbenzene-1,7-octadiene copolymer. After the calcium ion exchange, the final product was washed with ethanol and water.

### Example 2

### Powder Formulation

The following example describes a powder formulation of the invention. All the excipients used in the powder formulation are available from commercial sources and meet the specifications defined in the current compendial monograph. Suppliers include Cabot, Colorcon, Spectrum and FONA. The active pharmaceutical ingredient (API) was cross-linked (calcium 2-fluoroacrylate)-divinylbenzene-1,7-octadiene polymer, prepared as described in Example 1. The powder formulation was prepared by loading polymer with water and sorbitol as described above. Thereafter the stabilized polymer batch was split in about half, which was mixed with the other ingredients described below. The mixture was screened and then the second about half of the stabilized polymer was added to the mixture. The entire mixture was thoroughly mixed and then screened again.

The powder formulation may be reconstituted with water at, for example, a ratio of 1:5 (powder/water), such that a 15g dose of API will be 75 mL of water. On the other hand, the formulated powder can be mixed with soft foods such as applesauce, yogurt or pudding for administration. The powder was packaged in 60 cc wide mouth, white high density polyethylene (HDPE) bottles with 15 g of the polymer of Example 1 per bottle.

TABLE 1

| Ingredient | function | grams | Wt. % |
|---|---|---|---|
| polymer | API | 15 | 56.97% |
| sorbitol | API stabilizer | 6.2 | 23.55% |
| Water | API stabilizer | 4.6 | 17.47% |
| xanthan gum | Suspending agent | 0.185 | 0.70% |
| colloidal silicon | Glidant | 0.248 | 0.94% |
| dioxide | | | |
| Yellow dye | Coloring agent | 0.005 | 0.02% |
| titanium dioxide | Opacity | 0.09 | 0.34% |
| TOTAL | | 26.328 | 100.00% |

A second powder formulation was prepared having an antimicrobial agent added. The ingredients for the second powder formulation are listed in Table 2.

TABLE 2

| Ingredient | function | grams | Wt. % |
|---|---|---|---|
| polymer | API | 15 | 56.89% |
| sorbitol | API stabilizer | 6.2 | 23.52% |
| Water | API stabilizer | 4.6 | 17.45% |
| xanthan gum | Suspending agent | 0.185 | 0.70% |
| colloidal silicon | Glidant | 0.248 | 0.94% |
| dioxide | | | |
| Dye or dye | Coloring agent | 0.005 | 0.02% |
| blend | | | |
| methylparaben | Antimicrobial | 0.028 | 0.11% |
| propylparaben | Antimicrobial | 0.009 | 0.03% |
| titanium dioxide | Opacity | 0.09 | 0.34% |
| TOTAL | | 26.365 | 100.00% |

When introducing elements of the present invention or the embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and methods without departing from the scope of the invention, it is intended that all matter contained in the above description shall be interpreted as illustrative and not in a limiting sense.

1. A powder formulation comprising a potassium-binding active agent, a suspending agent, and a glidant, wherein at least about 40 wt. % active agent is present in the composition based on the total weight of the solid components, and wherein the powder is in the form of substantially spherical beads.

2. A powder formulation comprising a potassium-binding polymer, water, a suspending agent and an antimicrobial agent, wherein the amount of water does not prevent the powder particles from freely flowing.

3. The powder formulation of claim 1 wherein the active agent is a potassium-binding polymer.

4. The powder formulation of claim 2 wherein the potassium-binding polymer is a crosslinked aliphatic carboxylic polymer.

5. The powder formulation of claim 2 wherein the suspending agent comprises xanthan gum, polycarbophil, hydroxypropyl methyl cellulose, povidone, methylcellulose, dextrin, sodium alginate, (poly)vinyl alcohol, microcrystalline cellulose, a colloidal silica, bentonite clay, or a combination thereof

6. The powder formulation of claim 5 wherein the suspending agent comprises xanthan gum.

7. The powder formulation of claim 2 further comprising a glidant.

8. The powder formulation of any one of claim 1 wherein the glidant comprises colloidal silicon dioxide, aluminum silicate, talc, powdered cellulose, magnesium trisilicate, silicon dioxide, kaolin, glycerol monostearate, metal stearates such as magnesium stearate, titanium dioxide, starch, or a combination thereof.

9. The powder formulation of claim 8 wherein the glidant comprises colloidal silicon dioxide.

10. The powder formulation of claim 1 further comprising an antimicrobial agent.

11. The powder formulation of any one of claim 2 wherein the antimicrobial agent comprises α-tocopherol, ascorbate, methylparaben, ethylparaben, propylbaraben, butylparaben, pentylparaben, hexylparaben, benzylparaben, chlorobutanol, phenol, sodium benzoate, benzalkonium chloride, benzethonium chloride, chlorobutanol, phenylethyl alcohol, or a combination thereof.

12. The powder formulation of claim 2 further comprising a stabilizing polyol.

13. The powder formulation of claim 12 wherein the stabilizing polyol comprises D-(+)arabitol, erythritol, glycerol, maltitol, D-mannitol, ribitol, D-sorbitol, xylitol, fructose, manitol, mannose, or a combination thereof

14. The powder formulation of claim 13 wherein the stabilizing polyol comprises D-sorbitol.

15.-18. (canceled)

19. The powder formulation of any one of claim 2 comprising at least about 50 wt. % active agent based on the total weight of the formulation.

20.-29. (canceled)

30. The powder formulation of claim 2 wherein the powder is contained in a single dose or multi-dose container.

31. The powder formulation of claim 2 wherein the powder flows freely.

32. The powder formulation of claim 2 wherein the particles are in the form of substantially spherical particles.

33.-41. (canceled)

42. The powder formulation of claim 2 wherein the potassium-binding polymer comprises structural units corresponding to Formulae 1 and 2, Formulae 1 and 3, or Formulae 1, 2, and 3, wherein
Formula 1, Formula 2, and Formula 3 are represented by the following structures: 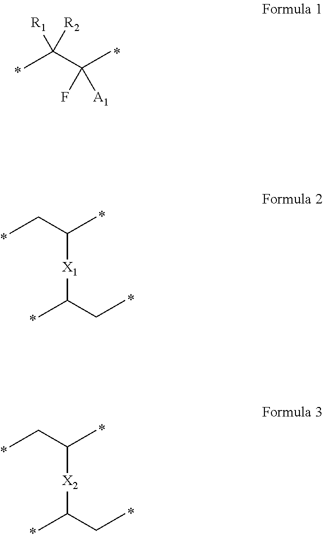
wherein
R₁ and R₂ are each independently hydrogen, alkyl, cycloalkyl, or aryl;
A₁ is a carboxylic, phosphonic, or phosphoric moiety;
X₁ is an arylene moiety; and
X₂ is an alkylene, ether, or amide moiety.

43. The powder formulation of claim 2 wherein the potassium-binding polymer comprises a product of a reaction of (1) a polymerization mixture comprising a polymerization initiator and monomers of either (i) Formulae 11 and 22, (ii) Formulae 11 and 33, or (iii) Formulae 11, 22, and 33; and (2) a hydrolysis agent; and
Formula 11, Formula 22, and Formula 33 are represented by the following structures: 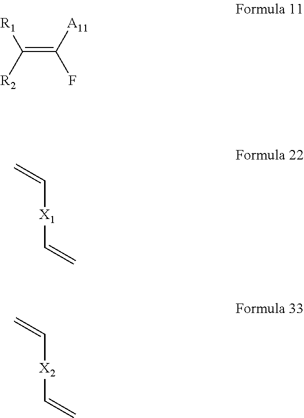
wherein
R₁ and R₂ are each independently hydrogen, alkyl, cycloalkyl, or aryl;
A₁₁ is a protected carboxylic, phosphonic, or phosphoric moiety;
X₁ is an arylene moiety; and
X₂ is an alkylene, ether, or amide moiety.

44.-64. (canceled)
